(19) United States
(12) Patent Application Publication
(10) Pub. No.: US 2017/0147765 A1
(43) Pub. Date: May 25, 2017

(54) SYSTEMS AND METHODS FOR TREATMENT OF STROKE

(71) Applicant: Penumbra, Inc., Alameda, CA (US)

(72) Inventor: Brijesh P. Mehta, Miami Lakes, FL (US)

(73) Assignee: Penumbra, Inc., Alameda, CA (US)

(21) Appl. No.: 15/356,248

(22) Filed: Nov. 18, 2016

Related U.S. Application Data
(60) Provisional application No. 62/257,400, filed on Nov. 19, 2015.

(51) Int. Cl. G06F 19/00; A61B 5/11; G06Q 10/06; A61B 5/00

(52) U.S. Cl. CPC G06F 19/327; A61B 5/0022; A61B 5/1113; A61B 5/746; A61B 5/681; G06Q 10/0633; G06F 19/321; G06F 19/325

(57) ABSTRACT

A system for treatment of ischemic stroke provides a stroke treatment workflow plan defining series of diagnostic actions and therapeutic actions to be performed at locations within a health care facility identified by beacons detectable by proximity sensors that travel with the patient. A first communications device having wireless communications capabilities receives a signal from a proximity sensor and typically transmits data to a second communications device having a visible timer and configured to receive data from and send data to other wireless communications devices. When the a patient undergoes diagnosis and treatment via the workflow plan, the system tracks the location of the patient within the workflow plan and the time at which the patient is at each location, and records the location of the patient and the time of the location within the workflow plan.

### CROSS-REFERENCE TO RELATED APPLICATIONS

This application claims the benefit of Provisional No. 62/257,400, filed Nov. 19, 2015, the entire content of which is incorporated herein by reference.

### FIELD OF THE INVENTION

The present invention relates generally to medical systems and procedures, and management of medical procedures and personnel. Specifically, the invention relates to treatment of ischemic stroke. The invention further relates to methods of administering technology intensive medical care and managing multidisciplinary teams that perform complex, life-saving medical procedures within restrictive time constraints. The invention also relates to business methods for evaluating a treatment facility's effectiveness in handling complex medical procedures, and for providing quantified consultation to a treatment facility for improvement of the care provided by the facility.

### BACKGROUND OF THE INVENTION

Stroke is a significant cause of disability and death, and is a growing problem for global healthcare. More than 700,000 people in the United States alone suffer a stroke each year, and of these, more than 150,000 people die. Of those who survive a stroke, roughly 90% will suffer long term impairment of movement, sensation, memory, or reasoning, ranging from mild to severe. The total cost to the U.S. healthcare system is estimated to be over $50 billion per year.

Stroke may be caused from a rupture of a cerebral artery (referred to as a “hemorrhagic stroke”), or by a blockage or occlusion in a cerebral artery resulting from a thromboembolism (referred to as an “ischemic stroke”). Roughly 80% of strokes are classified as ischemic. When a patient experiences an ischemic stroke, the occlusion prevents blood flow to vital brain tissue, thereby depriving the tissue of oxygen, causing nerve cell damage and potentially cell death. Among patients experiencing a stroke due to a large vessel occlusion, approximately 1.9 million nerve cells (or neurons) are at risk for irreversible injury every minute that elapses, until blood flow is restored. Providing rapid and effective diagnosis and treatment of stroke is therefore vital for protecting and restoring patient health.

Health education aims to alert the public to the signs and symptoms of stroke, and the vital importance of getting immediate medical assistance when a stroke is suspected. Once medical assistance is sought, either through an emergency response by paramedics or arrival in a hospital emergency room, a series of examinations and tests is initiated. Stroke is typically diagnosed by first using patient interview and examination, including protocol to detect one-sided weakness or paralysis, speech difficulty, or other common symptom of stroke. In order to objectively quantify the impairment caused by a stroke, a healthcare provider will use the National Institutes of Health Stroke Scale, or NIHSS. A protocol including 11 items of inquiry, the sum of the patient's score for each inquiry is calculated in order to assign a score reflecting the severity of the stroke. Further, if a stroke is suspected as a result of the interview and physical exam, diagnostic imaging such as CT scan, MRI, ultrasound, or some combination is then performed in order to definitively diagnose a stroke. The imaging process also determines the location of an occlusion, and prepares clinicians for treating the clot. All of the foregoing requires the recording of information and the communication of test results to treating physicians. Therefore, beginning with emergency responders, admissions personnel, physicians, nurses, diagnostic imaging technicians, and other support personnel, the diagnostic process alone involves numerous medical professionals, and requires rapid and precise communication of results throughout the protocol, all accompanied by the time pressure presented in a case of acute stroke.

Following diagnosis of acute ischemic stroke, there are currently two FDA approved therapies: intravenous administration of a drug referred to as tissue plasminogen activator, or tPA, and mechanical thrombectomy performed under fluoroscopic imaging. Approved use of tPA is limited to within three hours of symptom onset, while mechanical thrombectomy may be deployed within up to eight hours. In view of the time constraints for safe administration of tPA and the rapid loss of neurons suffered during stroke, the American Stroke Association (ASA) guidelines recommend administration of intravenous tPA within 60 minutes of time of arrival to the hospital.

If intravenous tPA is not an option for treatment, mechanical thrombectomy may be the desired course of therapy. Mechanical thrombectomy typically involves the use of an intravascular device such as a catheter. The distal end of an interventional catheter is introduced via a remote incision site (typically in the groin), and tracked to the site of the occlusion. The therapy typically involves aspiration, and may utilize additional interventional devices, such as a clot remover or separator, which is mounted to the distal end of the catheter. Recent randomized control trials show that rapid reperfusion is associated with favorable clinical outcomes. (See Stroke 2015; 46:3020-3035). Formulated from these studies are goal times of “picture-to-puncture” in less than 60 minutes, and “door-to-device” within 90 minutes. Needless to say, diagnosing and administering stroke treatment within these guidelines requires efficient work and communication among a large team of medical professionals, including emergency physicians, nurses, neurologists, radiologists, neurointerventionalists, and catheter lab staff. In order to consistently perform at a high level in delivery of acute stroke therapies and remain compliant with the ASA/Joint Commission recommendations, renewed focus has been placed on tracking time intervals during the in-hospital stroke processes in order to evaluate and to optimize workflows.

However, stroke treatment coordinators and quality improvement specialists currently manually track specified treatment metrics, and only through retrospective data extrapolation from Electronic Health Records (EHR) and from paper charts. Existing hardware and software solutions available in the market still require significant data entry during the time-sensitive stroke treatment procedures. These methods of data collection are disruptive, time consuming, susceptible to error, and lack the instructive benefit of rapid feedback following the conclusion of a case. Consequently, these methods are less than advantageous to continuous process improvement. Other attempts to improve the speed with which hospitals diagnose and treat stroke include pre-hospital notification from emergency personnel to stroke centers, but do not provide a comprehensive solution to the challenges presented. There remains a need to effectively track, log, and analyze protocol metrics. Moreover, there remains a need to establish and enhance immediate communication of test and imaging results among all team members simultaneously.

The aforementioned shortcomings in the prior art are also applicable to treatment of conditions other than stroke. For example, many of the same needs arise in the context of treatment of cardiac arrest, myocardial infarction, epilepsy, and childbirth. Therefore, a desirable solution to stroke treatment may have significant utility in many other treatment contexts.

A desirable solution should be easy to implement, and customizable to fit the hospital's existing procedures. The system should be HIPAA compliant, secure, and include reliable, automated capture of checkpoint metrics. The system and methods should provide automatic reporting of key data obtained during diagnosis and treatment.

### BRIEF DESCRIPTION OF THE DRAWINGS

FIG. 1 is a flow chart illustrating a series of exemplary actions in stroke treatment.

FIG. 2 is a schematic timeline reflecting American Stroke Association (ASA) timing guidelines for treatment of stroke.

FIG. 3 is a schematic illustration of some of the devices employed in systems and methods according to the invention.

FIG. 4 is a schematic illustration of some of the devices employed in systems and methods according to the invention.

FIG. 5 is a schematic illustration of a system and method according to the invention.

### DETAILED DESCRIPTION OF THE INVENTION

Some embodiments of the invention are described below. For clarity, not all features of each actual implementation are described in this specification. In the development of an actual system, some modifications may be made that result in an embodiment that still falls within the scope of the invention.

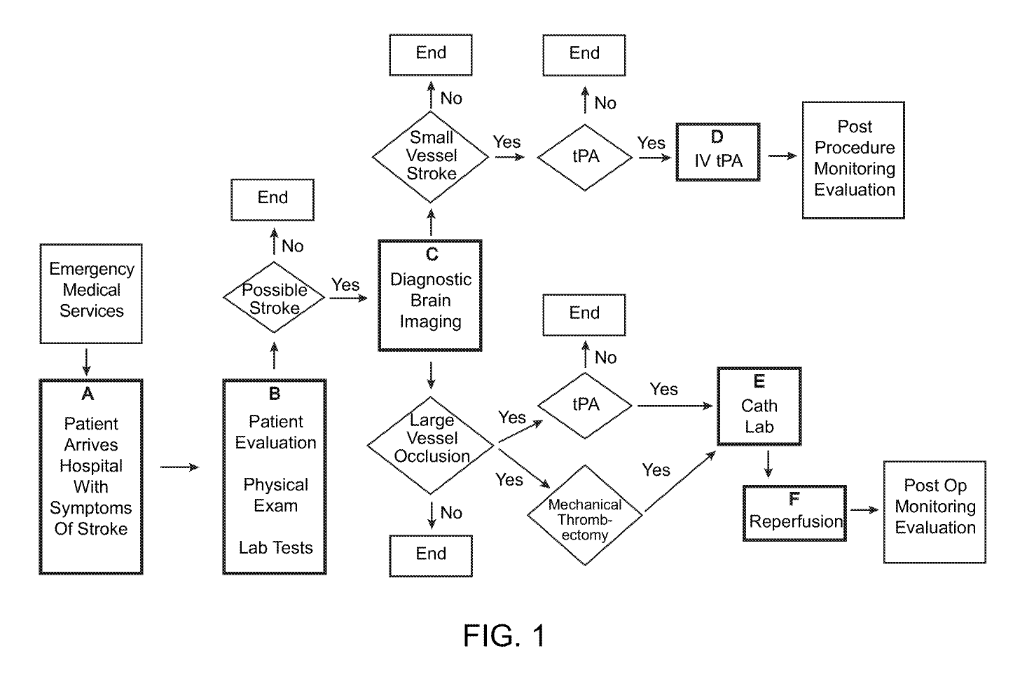

FIG. 1 is a flow chart that highlights some of the key actions that are taken during stroke treatment. Conceptually the decisions and actions form a workflow. The term “stroke treatment workflow” is used herein to refer to a progressive series of decisions made and actions taken in order to diagnose and treat stroke. It will be understood that FIG. 1 does not include the finer details of diagnosis and treatment. In fact, the events represented in FIG. 1 are very general, and could be broken down into multiple actions or checkpoints that take place within the workflow. For example, the physical exam includes numerous tasks, often undertaken by more than one care provider. The physical exam generates numerous data points which in turn are placed into a diagnostic matrix or algorithm. Similarly, the patient interview may include small tasks performed by a patient (such as, for example, raising both arms), taking a medical history, and other detailed tasks. The actions reflected in FIG. 1 are greatly simplified for the purposes of demonstration and clarity. Further, it will be understood that while the example of FIG. 1 is focused on stroke treatment, a comparable treatment workflow may be useful in treating other conditions, by substituting some of the key parameters and steps in stroke treatment with the key steps in the protocol for treating other conditions. Some of these key steps are referred to generically as “intervention”, “interventional measures”, “therapeutic intervention”, or comparable term.

Beginning at the far left of the figure, the first event in the workflow is referred to as “Emergency Medical Services”. During this first event, emergency responders such as, for example, paramedics respond to an emergency call for medical assistance. Emergency medical service providers evaluate the patient and, if warranted, transport the patient to a hospital. During the evaluation by emergency medical services, several data points are generated related to the patient and the patient's symptoms. (It will be noted however that in some instances, a patient is transported directly to a hospital without the intervention of emergency medical services.) Following this optional initial intervention, box A in the workflow illustrated represents the patient's arrival at a hospital. When a patient arrives at a hospital and is suffering symptoms of stroke, a series of events is initiated in order to diagnose and treat stroke. This series of events is labeled B in FIG. 1. As illustrated in B, the patient is interviewed, and a physical exam is administered that includes tests for neurological deficit. In addition, blood is drawn and laboratory tests are performed in order to detect indicators of stroke. If acute ischemic stroke is suspected, the process continues to C in the illustration of FIG. 1, and the patient undergoes diagnostic imaging tests such as a CT scan or MRI. The imaging conclusively determines whether there is an occlusion of blood flow, locates the occlusion, and reveals additional diagnostic details of the occlusion which are important for formulating a treatment plan. Importantly, the imaging determines whether the occlusion is a “Small Vessel Stroke”, or a “Large Vessel Occlusion”, both of which are noted as options in FIG. 1. If it is determined that neither of these conditions exists, for the purposes of the illustration, the workflow ends.

If a “Small Vessel Stroke” is diagnosed, a determination is made whether the patient is a candidate for intravenous tPA. If tPA is selected as the optimal treatment, the patient is prepared and if necessary, moved to a suitable location for the administration of tPA, represented by box D in FIG. 1. If, however, it is determined that administration of tPA is not within safe time limits, or is otherwise contraindicated, then tPA will not be administered, and for the purposes of the illustration of FIG. 1, the workflow ends.

If a “Large Vessel Occlusion” is diagnosed, a determination is also made whether the patient is a candidate for intravenous tPA. However, in the case of a “Large Vessel Occlusion”, a determination is also made whether the patient will undergo mechanical thrombectomy. If mechanical thrombectomy is a desired course of treatment, the patient proceeds to a catheterization lab, or “cath lab”, and is prepared for a percutaneous catheter procedure under fluoroscopic visualization (E). Mechanical thrombectomy and intravenous tPA may be administered in combination. Further, mechanical thrombectomy may involve one or more various alternative devices and methods. The goal of all of the available devices and methods is reperfusion of the affected vessel, and restoration of blood flow (F). Following reperfusion, the patient is continually monitored and evaluated. Recovery benchmarks are recorded and analyzed.

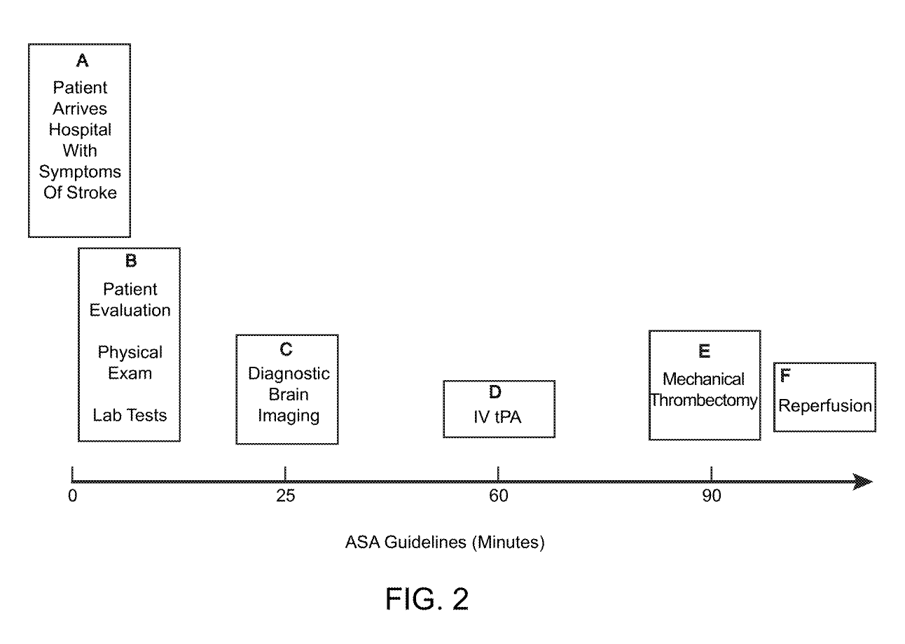

FIG. 2 is a schematic illustration of a timeline of some of the key events of FIG. 1. The points along the axis of FIG. 2 reflect the desired goal times by which it is desirable, according to American Stroke Association (ASA) guidelines, to achieve some of the major workflow tasks illustrated in FIG. 1. Beginning at the left hand side of FIG. 2, the time of arrival at the hospital (A) is considered the start time, or 0 minutes. This start point is also nicknamed “Door” time in stroke treatment protocol jargon. In the following minutes, the numerous diagnostic tasks (prior to imaging) are performed (B). Diagnostic brain imaging (C) is ideally performed within 25 minutes of arrival. And following conclusive imaging of an acute stroke, administration of intravenous tPA (D), is ideally initiated within 60 minutes of hospital arrival. Also according to the stroke guidelines discussed above, 90 minutes is the goal by which mechanical thrombectomy is performed (E), followed by reperfusion (F). Patient monitoring, periodic neurochecks and the computation of NIHSS scores continue in the hours following treatment. Treatments other than stroke may utilize a comparable timeline, but may include other key timing guidelines associated with key procedural steps that are appropriate for the particular disease, condition, or medical event.

The invention herein includes systems and methods for treatment of stroke, myocardial infarction, cardiac arrest, or other emergency medical treatment. The system includes a treatment workflow plan, and an interrelated group of devices and methods designed to be integrated into the workflow, with the goal of accomplishing critical tasks within the timing guidelines illustrated in FIG. 2 or other applicable timing guidelines. The invention disclosed herein provides automated tracking of a patient's progress through the workflow of FIG. 1, and provides real-time updates to all of the multidisciplinary team members as the patient progresses through the stroke treatment workflow, while continually tracking actual elapsed time. In addition, the system furnishes “push” notifications to various team members as the patient progresses through the protocol, summoning members to corresponding work stations, and alerting members to particular action items. Further, the system manages the substantial data that is generated at each point in the protocol, up to and including the conclusion of the case, thereby providing immediate feedback to the multidisciplinary team. Still further, the system compares the data with previous cases, immediately highlighting bottlenecks in the workflow, thereby focusing and streamlining efforts of the hospital to improve workflows. The system may even compare cases handled by competitor hospitals, and provide quantitative success rates that hospitals may use to promote their services. And still further, the system automatically exports data to spreadsheets and electronic health records (EHRs), enabling evaluation of the data and improvement of workflow efficiencies. Finally, the system includes a dashboard available online and through a mobile app providing immediate process summary upon completion of each stroke case, highlighting achievements and areas for improvement

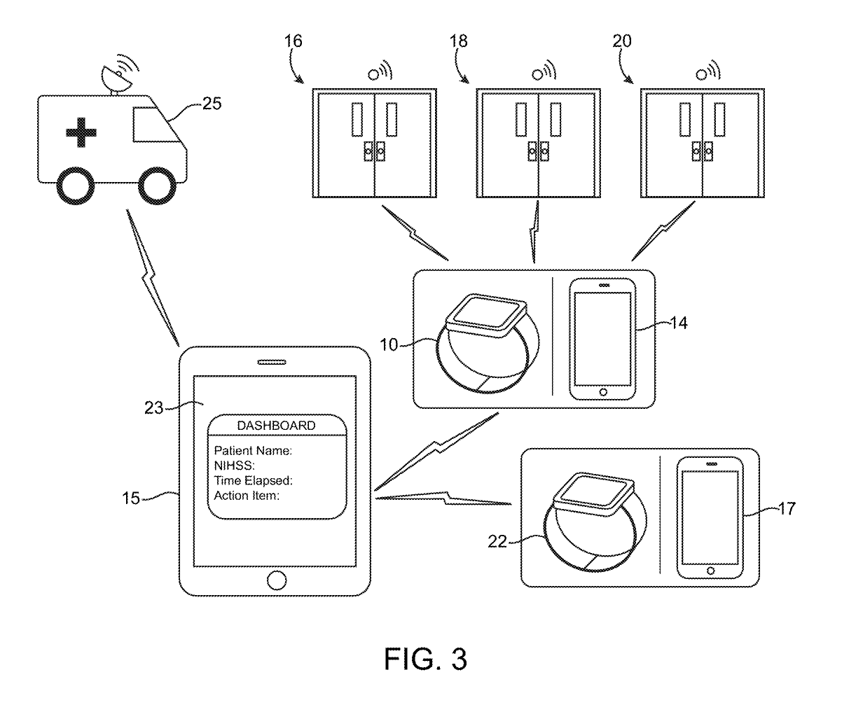

The systems and methods according to the invention incorporate known devices, and employ hardware and software customized for the system. The principle devices suitable for use with the invention are illustrated in FIG. 3, and begin first with an optional global positioning system (GPS) 25, located within an emergency medical services vehicle, and any communications devices used by personnel in the vehicle (not pictured). Location of the vehicle and any diagnostic information obtained by emergency services may be transmitted to other communications devices used in the system. The key devices in the illustration of FIG. 3 also include a “smart” watch 10, a “smart” phone 14, and beacons 16, 18 and 20. The term “smart watch” is used herein to refer to a computerized mobile device that provides timekeeping and extensive additional functions, has the capability to run mobile applications, and is designed to be worn on the wrist. Several brands of smart watches are currently commercially available, and numerous will become available, that are suitable for use with the invention. The term “smart phone” is intended to refer to a mobile phone that utilizes an advanced mobile operating system which combines features of a personal computer operating system with communications capabilities, high resolution touch screen display, WiFi connectivity, the ability to accept sophisticated applications, and other features useful for mobile or handheld use. Numerous brands of smart phones, such as Apple iPhone, Android, Samsung, and others are currently commercially available, and additional smart phones will become commercially available, and are suitable for use with the invention. The term “beacon” is used herein to refer to an electronic, signal emitting proximity sensor, the beacon equipped to emit a unique identifier that is received by a mobile communications device such as a smart watch having compatible software. “Beacon” may also include or alternatively refer to radiofrequency identification tags, both transmitting and receiving, used for tracking the movement of items or persons.

Additional devices that may be incorporated into the system include additional smart watches, which may be worn by medical personnel, one or more tablet computers (such as, for example, an iPad), laptop computers, and a “smart TV”, such as Apple TV. A system or method according to the invention may employ any number of the aforementioned devices that are capable of receiving, transmitting, displaying and recording data. The aforementioned devices are collectively referred to herein as “communications devices” or “wireless communications devices”. Moreover, many of the mentioned communications devices may be interchangeable with one another within the systems and methods disclosed herein.

The smart watch 10 is to be worn by a patient, and is linked by its software to smart phone 14. Alternatively, or in addition, smart watch 10 may be linked to a computer tablet or laptop computer (not pictured). (The smart watch may also be replaced by an alternative communications device, such as, a smart phone.) In the alternative, smart watch 10 may be replaced by a radiofrequency identification tag, which may be included in a patient wrist bracelet, or otherwise closely associated with the patient. Beacons (or radiofrequency signal emitters) 16, 18 and 20 are located at or near the entry and/or exit of any of a number of designated sites within a hospital that are locations to which a patient is brought during stroke treatment. These sites may include an emergency room, a CT scan, MRI, or comparable imaging suite, a cath lab, and other locations. Beacons 16, 18 and 20 communicate wirelessly with smart watch 10. Additional beacons (not pictured) may be mounted at additional or alternative sites as customized by a hospital. An example of suitable beacons are iBeacons, (a protocol standardized by Apple, https://developer.apple.com/ibeacon/) which use Bluetooth Low Energy (LE) proximity sensing to transmit a universally unique identifier that is picked up by a compatible app or operating system. An example of a radiofrequency identification tag is of a type used in athletics for tracking the movement of an athlete, or used by commercial carriers to track movement of a shipped package. The identifier can be used to determine the physical location of a device (here, smart watch 10), or trigger a location-based action. Smart watch 10 in turn can communicate this information, or transmit this data, to smart phone 14. Smart phone 14 can in turn upload the information to another smart watch 17, smart phone 22, a tablet 15, a smart TV and/or any device that may display online dashboard 23. The term dashboard is used herein to refer to a software-based control panel for the applications used by the system. The dashboard may display data, both singularly and in graph or chart form, time elapsed, actions needed, and other desired interactive elements.

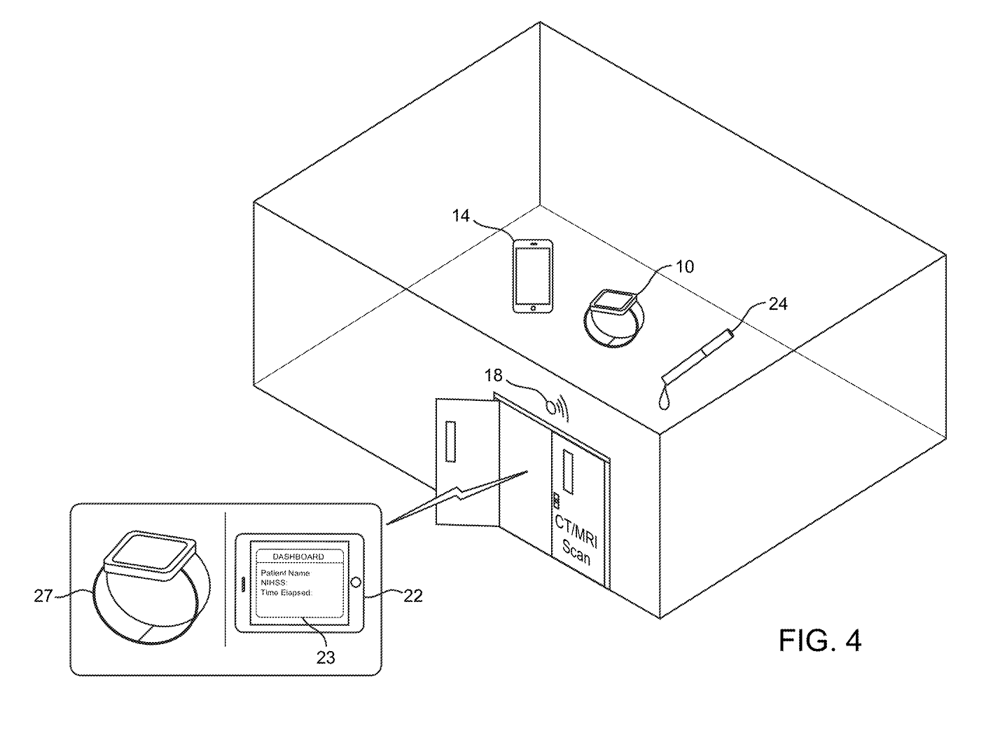

FIG. 4 illustrates an additional device that may be incorporated into the system. Beacon wand 24 may be located within or near imaging suite 26. Additional beacon wands may similarly be located within or near each of the preceding locations, and additionally or alternatively at other locations as customized by a particular treatment center. Beacon wand 24 communicates wirelessly with smart watch 10 and smart phone 14 when brought near the device or devices. Beacon wand 24 communicates to automatically register patient location contextual steps in the workflow, and to log specific time points, such as CT scan start, CT scan completion, initiation of tPA administration, etc. This data is transferred to smart watch 10 and smart phone 14, to continue the tracking of critical information regarding the patient's test results, overall condition, and the patient's progress through the stroke treatment workflow.

The system preferably includes turnkey hardware/software. The software preferably is user friendly, includes a simple user interface and requires minimal lead-in training. It must be HIPAA compliant, secure, and utilize data encryption. The smart watch 10 and smart phone 14 permit rapid data entry by physicians and nurses through a simple user interface (e.g., patient age/name, NIHSS score, etc.), during the treatment workflow. The system should include the ability to share case summary and dashboard metrics with emergency management systems (EMS) as part of a virtual poster; to compare process metrics with other sites utilizing the platform around the world; and backend data analytics software for quality improvement and research.

The system further includes a stroke process app designed for a smart phone 14 that displays time lapse, and also receives information (such as patient location, etc.) via the smart watch 10, as the patient progresses through the stroke treatment process pathway. The smart phone 10 (such as, for example, an iPhone 6 plus), may be stationed on the patient stretcher, permitting team members to view time lapse from arrival at the hospital, and to input data. The highly visible display of time lapse conveys the continuing sense of urgency throughout the protocol. The various phases and time intervals of the workflow can also be displayed as each step in the process is completed, keeping all team members aware of the patient's progress and the hospital's efficiency. Location specific features in the smart phone software will allow entry of contextual data such as age, NIHSS score, LSW [spell out] time, tPA administration time, puncture time/devices used/reperfusion time/TICI [spell out] score, via beacons prompting next steps along the workflow programmed into the app. All fields should be easily customizable based on hospital preferences.

The smart watch 10 (such as, for example, Apple Watch) is worn by the patient, and utilizes wireless communication to receive location identifying information for the beacons positioned along the stroke treatment process workflow. The smart watch 10 provides communication location status updates to the smart phone 14 after receiving location signal information from the proximity beacon 18. A unique identifier of the beacon can be programmed into the app for location input or action input as mentioned above. The smart watch 10 may additionally behave as a key that unlocks each phase of the stroke process on the smart phone 14 after receiving location pings from the beacon(s) 16, 18 or 20.

The system also incorporates a mobile app for smart phones used by treating physicians and nurses, who would receive push notifications of the stroke workflow as the patient progress through the process. And upon completion of the stroke case, the data would be pushed immediately to an online dashboard, with options to export to the hospital's EHR for seamless documentation.

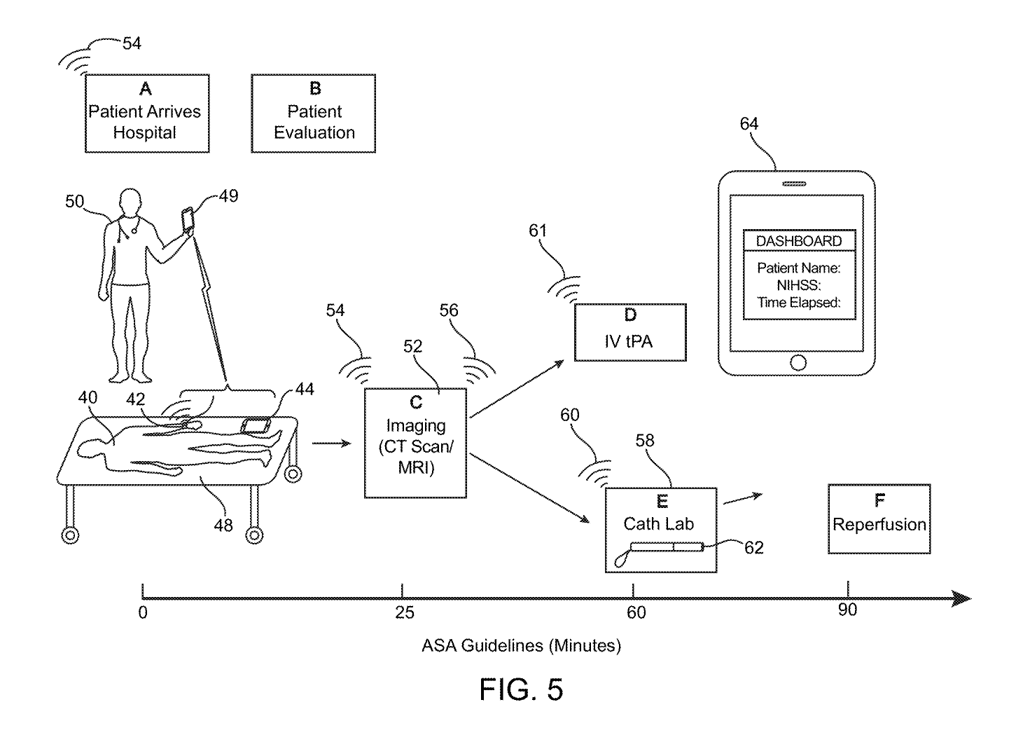

Turning now to FIG. 5, an example of a system and method according to the invention will be illustrated. Firstly, a stroke patient 40 arrives in the hospital emergency room (ER); a smart watch 42 is placed upon the patient's wrist, and a smart phone 44 is assigned to the case. A beacon 46, either within a wand or otherwise located in the ER, sends a signal to the smart phone to activate a stroke alert app. The ER arrival time is automatically logged, and smart phone 44 is placed on the stretcher 48 in a manner for high visibility of the clock, and for easy access by clinicians. Basic information, such as patient name, LSW time, NIHSS, neurological deficits, etc. is entered into the phone app. The phone sends push notifications to smart phones 49 of stroke team members such as physician 50, and logs the stroke alert activation time. These push notifications or alerts can be simultaneously transmitted to all other members of the stroke treatment team, thereby enhancing prompt communication to all care providers, and improving timeliness and overall care.

The patient is rolled to the CT scanner 52, smart phone 44 all the while displaying the time lapse. Upon patient entry into the CT scanning suite, a beacon 54 positioned at the door sends a location signal to watch 42, which then communicates with phone 44 for logging the CT entry time. The CT phase information is entered into the phone app via a separate beacon (not pictured), positioned in the scanner area and rads reading room, alerting the app to prompt the next steps. Examples of the data at this point include reporting that the CT is completed, and diagnostic information gleaned from the CT, such as hyperdense sign vs bleed, aspects score, tPA time, LVO Yes/No [spell out]. The patient is then rolled out of the CT room, and beacon 56 sends a signal for the departure time to smart watch 42. All of the foregoing can be uploaded to an online dashboard and displayed on a device such as smart TV 64.

If the decision is made to perform a mechanical thrombectomy, the patient is then transported to the cath lab 58. The smart phone 44 remains with patient 40, and upon patient entry into the suite, the beacon 60 positioned at the door sends a location signal to watch 42, which then communicates with the phone 44 for logging angiography suite entry time. Cath lab phase data is entered into the phone app via a visual process map posted in the suite with a separate beacon 62 that would prompt the user for puncture time, devices used, reperfusion time, TICI score, etc. Upon case completion, all information is immediately available on a dashboard 64 for research/QI or sharing with local EMS and hospital staff. The data can therefore be immediately evaluated, and problem areas within the workflow can be pinpointed for improvement.

The foregoing examples are not intended to limit the scope of the invention. All modifications, equivalents and alternatives are within the scope of the invention.

What is claimed is:

1. A system for treatment of ischemic stroke, the system comprising:
a stroke treatment workflow plan including a series of diagnostic actions and therapeutic actions to be performed at designated locations within a health care facility;
a first communications device including a software application and wireless communications capabilities, the communications device capable of receiving a beacon signal from a proximity sensor and capable of transmitting data to a second communications device;
a second communications device including a software application and a visible timer, the second communications device having wireless communications capability enabling it to receive data from and send data to other wireless communications devices;
a plurality of signal beacons, each signal beacon located at a designated location within the workflow plan, the signal beacons capable of wirelessly sending a location specific signal to the first communications device;
wherein when a patient undergoes diagnosis and treatment via the workflow plan, the first communications device is worn by or otherwise closely associated with the patient, the system tracks the location of the patient within the workflow plan and the time at which the patient is at each location, and records the location of the patient and the time of the location within the workflow plan.

2. The system according to claim 1, wherein the system further comprises a third communications device, and the second communications device transmits the time and the location to the third communications device, and the data is recorded for analysis.

3. The system according to claim 1, wherein the system further comprises a third communications device, and the second communications device transmits alert notifications to the third communications device to alert a user to perform a task.

4. The system according to claim 1, wherein the system further comprises a third communications device, and the first communications device, the second communications device, and the third communications device all include a software application linking the first communications device, the second communications device, and the third communications device to incorporate a stroke treatment workflow, and to track time, a patient's location, diagnostic data, and to provide alert notifications to a system user.

5. The system according to claim 4, wherein the system further comprises customizable software and an online dashboard.

6. The system according to claim 1, wherein the system further comprises at least one beacon wand, wherein said beacon wand, when brought in physical proximity to the first communications device, transmits data to the second communications device.

7. The system according to claim 6, wherein the data comprises location information, diagnostic information, or both.

8. The system of claim 1, wherein the locations and the timing are automatically uploaded to a software application for review.

9. The system of claim 1, wherein the first communications device and the second communications device are selected from the group consisting of a smart watch, a smart phone, a laptop computer, a tablet computer, and a smart TV.

10. A method of treatment of ischemic stroke, the method comprising the steps of:
formulating a treatment work flow plan, the plan including suitable diagnostic and treatment actions;
providing a first communications device, a second communications device, and a plurality of proximity sensor beacons that each emit a unique identifier signal;
equipping the first communications device with a software application that recognizes each signal and assigns a location to each signal and the time of receipt of each signal, and that links the first communications device to the second communications device in order to transmit information from the first communications device to the second communications device.

11. The method according claim 10, wherein the method comprises the additional step of equipping the first communications device or the second communications device or both with a software application that incorporates the treatment work flow plan and the capability to send alert notifications.

12. The method according to claim 10, wherein the method comprises the additional step of placing the first communications device on or near a patient's body and the second communications device in close proximity to the patient, and initiating the timer and software applications to track the progress of the patient through the work flow plan.

13. The method according to claim 10, wherein the method includes the additional steps of providing a third communications device, and equipping the third communications device with a software application that enables the third communications device to receive information from the second communications device.

14. The method according to claim 13, wherein the information received by the third communications device from the second communications device includes the time of administration of a diagnostic test, or the time of administration of a therapeutic treatment, or both.

15. The method according to claim 14, wherein the information received by the third communications device from the second communications device includes an alert to a medical care provider to take an action.

16. The method according to claim 11, wherein the first communications device, the second communications device, and the third communications device are selected from the group consisting of a smart watch, a smart phone, a tablet computer, a laptop computer, and a smart TV.

17. A system for managing a healthcare facility's capability to treat patients, the system comprising:
a treatment workflow plan through which a patient exhibiting symptoms proceeds upon arrival at the facility, the workflow plan having specified checkpoints representing key diagnostic and/or therapeutic actions within the workflow plan;
a first communications device worn by a patient or placed in close physical proximity to a patient proceeding through the treatment workflow plan, the first communications device including software and wireless communications capability for receiving a proximity sensor signal and for transmitting data to a second communications device;
a second communications device including customizable software and a visible timer, the second communications device having wireless communications capability enabling it to receive data from and send data to other wireless communications devices;
a plurality of proximity sensors located within the facility at specified checkpoints within the workflow plan, the proximity sensors in wireless communication with the first communications device and capable of sending a location specific signal to the first communications device;
a third communications device selected from the group consisting of: a smart phone, a smart watch, a computer tablet, a laptop computer, a smart TV or other computer;
whereby as a patient proceeds through the workflow plan, the first communications device tracks the location of the patient within the workflow plan, the second communications device records the location of the patient and the time of the location within the workflow plan; the second communications device transmits the time and the location to at least one additional communications device, and the data is recorded for analysis.

18. The system according to claim 17, wherein the treatment workflow plan incorporates goal times for a patient's arrival at the specified checkpoints, and the data includes actual elapsed time at the patient's arrival at the specified checkpoints, and the data is compared to the goal times.

19. The system according to claim 17, wherein the workflow plan includes diagnostic imaging, and the specified checkpoints include arrival at the facility and arrival of the patient at the facility's diagnostic imaging location.

20. The system according to claim 19, wherein the specified checkpoints include initiation of interventional measures.

21. The system according to claim 17, wherein the workflow plan includes diagnostic imaging, and the specified checkpoints include arrival at the facility, arrival of the patient at the facility's diagnostic imaging location, and administration of therapeutic intervention.

22. A business method for providing consultation to a stroke treatment facility, the method comprising charging a fee to deploy a system for evaluating the facility's compliance with the American Stroke Association's guidelines for stroke treatment, the system comprising:
a stroke treatment workflow plan through which a patient exhibiting stroke symptoms proceeds upon arrival at the facility, the workflow plan having specified checkpoints representing key diagnostic and/or therapeutic actions within the workflow plan;
a smart watch worn by a patient proceeding through the stroke treatment workflow plan, the smart watch including customizable software and wireless communications capability for receiving a proximity sensor signal and for transmitting data to a smart phone;
a first smart phone including customizable software and a visible timer, the smart phone having wireless communications capability enabling it to receive data from and send data to wireless communications devices;
a plurality of proximity sensors located within the facility at specified checkpoints within the workflow plan, the proximity sensors in wireless communication with the smart watch and capable of sending a location specific signal to the smart watch;
at least one additional wireless communications device selected from the group consisting of: a second smart phone, a computer tablet, a laptop computer, or other computer;
whereby as a patient proceeds through the workflow plan, the smart watch tracks the location of the patient within the workflow plan, the smart phone records the location of the patient and the time of the location within the workflow plan; the smart phone transmits the time and the location to at least one additional communications device, and the data is recorded and uploaded to a spreadsheet for comparison to the American Stroke Association's guidelines.

23. A network of proximity sensors, one or more electronic identification tags, and one or more communications devices, the network for tracking a patient's progress through a treatment workflow plan and for communicating the patient's progress to the communications device or devices, the proximity sensors distributed in a hospital at selected locations, the selected locations corresponding to key locations within a treatment workflow plan, the electronic identification tag or tags worn by or otherwise closely associated with a patient undergoing treatment through the treatment workflow plan.

24. The network according to claim 23, wherein the network further includes a software application associated with the network.

25. The network according to claim 24, wherein the software application includes the capability of providing alerts to one or more communications devices when the patient has reached one or more selected points in the treatment workflow plan.

26. A method of providing a hospital with a patient treatment tracking system, the method including the steps of:
providing a treatment workflow plan comprising key location points;
equipping the hospital with a plurality of proximity sensors disposed at or near the key location points;
providing the hospital with at least one electronic identification tag;
providing the hospital with a plurality of communications devices including software linking the devices to the proximity sensors and the electronic identification tag.

27. The method according to claim 26, wherein the treatment workflow plan includes timing guidelines.

28. The method according to claim 26, wherein the software comprises the capability of sending data to at least one communications device, wherein the data is received from the proximity sensors, the electronic identification tag, from other communications devices, or some combination of the foregoing.

29. The method according to claim 26, wherein the software further includes the capability of sending alert notifications to one or more communications devices.
